(19) United States
(12) Patent Application Publication
(10) Pub. No.: US 2008/0070245 A1
(43) Pub. Date: Mar. 20, 2008

(54) Method for herd management of domestic ruminants to control infection due to mycobacterium avium subspecies paratuberculosis

(76) Inventor: Gilles Reza George Monif, Bellevue, NE (US)

Correspondence Address: Gilles R. G. Monif, M.D., 17121 Lakewood Drive, Bellevue, NE, 68123-3970, US

(21) Appl. No.: 11/521,540

(22) Filed: Sep. 15, 2006

(51) Int. Cl. C12Q 1/68; C12P 19/34

(52) U.S. Cl. 435/6; 435/91.2

(57) ABSTRACT

A method of herd management of domestic animals contingent upon selective use and interpretation of nesting Map nesting PCR technology and resulting data, including qualitative and quantitative fecal screening for Map DNA, nesting Map PCR of milk, and establishing the criteria by which animals are moved constructively with in the herd or out of the herd.

### BACKGROUND OF THE INVENTION

(1) Field of Invention

The present invention relates to a method of management of ruminants who are subjected to endemic infection due to Mycobacterium avium subspecies paratuberculosis (Map and) whose biological products enter the human food chain.

(2) Description of the Prior Art

Map infection of domestic and nondomestic ruminants is global in its distribution. Map is the recognized inducer of a chronic granulomatous gastrointestinal disease (Johne's disease) in dairy cattle. The estimated annual economic loss to the dairy industry approaches $1.5-2 billion. While estimates of herd prevalence are comparatively low, within large herds, up to 40% of cows my be infected. Within herds with high infection rates, removal for slaughter of all test-positive cows would create an economically unfeasible situation.

The national herd management response to combat Johne's disease has been to adopt a policy of testing and culling serologically highly positive animals. Standards for sensitivity and specificity of commercial enzyme-linked immunosorbent assays (ELISA) have been largely developed established using fecal culture-positive cattle. The use of fecal-positive cattle and not necropsy confirmed cattle presumes the infallibility of fecal identification which, while the gold diagnostic standard, fecal recovery techniques are not inclusive of all serologically positive infected cows.

Collins et al. evaluated five antibody detection tests for the diagnosis of bovine paratuberculosis using serum samples from 359 dairy cattle in seven paratuberculosis-free herds and 2,094 dairy cattle in seven Map-infected dairy herds.

Both the ParaCheck (Biocor, Omaha, Nebr.) and HerdCheck (IDEXX Laboratories Inc. Westbrook, Me.) ELISA tests done in accordance with manufacturers' instruction and interpreted as prescribed by the kit insert, identified less than 29% of fecal culture positive cows. A positive relationship could be shown between the number of mycobacterium in feces and ELISA positivity. With a low number of Map in feces, a mean of 13.3% of infected cows were ELISA positive. At progressively higher fecal culture scores, the mean percentage of positive antibody assays were 27.3% 54.9% and 78.4% respectively.

Map has been isolated from up to 8% in milk of subclinically infected animals. Giese and Ahrens isolated Map from up to 35% of clinically infected animals. The Marshfield Clinic retail milk study found viable Map in 2.8% of milk samples taken directly from grocery stores in three of the nation's five largest dairy states. Map DNA was demonstrated in 64% of the 702 pints of milk sampled. Using nesting PCR, Buergelt and Williams, while showing a general positive correlation between increasing Map ELISA reading and increased probability of detection of Map DNA in milk, demonstrated that cows with initial low positive ELISA, initial suspicious ELISA and negative ELISA reading could potentially have Map DNA detected in their milk.

Crohn's disease is a chronic granulomatous disease affecting about 600,000 individuals in the United States. The interest in herd management emanates from a growing body of circumstantial evidence indicating that Mapas is the etiological agent for Crohn's disease. Using polymerase chain reaction, Map DNA can be identified in 60-80% of gastrointestinal mucosal specimens taken from individuals with Crohn's disease. Map has been cultured from the breast milk of 2 out of 5 lactating women with Crohn's disease and from the blood of other afflicted individuals.

Johne's disease has three distinct phases: undetected, subclinical, and clinical. In the undetected phase, the organism is shedded into the environment through its feces; however, no serological evidence of infection can be identified with current commercial tests. In its subclinical phase, fecal shedding is intensive and now, evidence of an antibody response can be detected using a enzyme-linked immunosorbant assat (ELISA). Arbritary determined values group animals into one of four categories: negative, inclusive, positive and strong positive. Using a nesting PCR technology which can identify Map DNA with very great sensitivity, it can be shown that Map ELISA titer does not correlate well with Map shedding into milk (Table 1).

USDA supports herd screening as a means to control intra-herd dissemination of Map. Once data is introduced what identified a given cow as having a test value indicative of infection/disease, the implication is that that dairy cow should be removed from the herd. The net cost to cull and replace a Holstein dairy cow is between at least $1,000 and $1,500

The gold diagnostic standard for diagnosing Map infection/disease has been based upon fecal recovery using artificial culture media. The technique is highly specific, but is suboptimal in terms of sensitivity. These techniques using artificial culture media are not inclusive of all serologically positive infected cows. At necropsy, cows have been shown to have infection/disease and yet their fecal cultures failed to grow the organism. Beyond the issue of limited sensitivity, there is the time in which a culture must be observed before being deemed negative. Cultures may become positive as long as six months after inoculation. In very rare instances, cultures have been reputed to become positive between six months and one year.

In the past two years, culture on artificial media has been pragmatically replaced by the Bactec MGIT 960 Mycobacterial System. The test involves specialized liquid media, patent sensors. Its advanced fluorometric technology permits use of highly accurate detection of oxygen consumption. Suspected cultures are then tested using standard PCR technology which involves IS900 derived primers. The Bactec system requires purchase of a specifical designed incubator (cost range $39-45,000) as well as antibiotics The time before a culture is deemed negative is 49 days.

The inventor and collaborators have developed a unique set of PCR primary and nesting primers which allow highly specific and sensitive detection of Map DNA in both feces and milk, Reproduction based upon three USDA fecal challenge kits was at 97% sensitivity and 93% specificity. This technology is currently in patent pending status. It provides the necessary tools which, along with the inventors intellectual property, created the proposed herd management program for Map.

### SUMMARY OF THE INVENTION

The present invention is directed toward a sequence of management steps with options to enhance control over the introduction of Map into the human food chain while attempting to minimize the adverse economic impact threatening the dairy industry. The method steps include:

a) screening the herds using nesting PCR to detect fecal Map,
b) if lactating, test the milk using nesting PCR to detect Map in milk
c) if not lactating, doing quantitative real time PCR to determine the quantity of mycobacterium present,
d) removing all lactating cows with Map in their milk from the herd,
e) removing from the herd all heavy shedders, and
f) subjecting all light shedders to a therapeutic regimen now under analysis

One aspect of the present invention is identifying infected cows, independent of serological or clinical staging of infection/disease. If the number is low, the herd manager may decide to eliminate all infected animals or select animals with low shedding for potential arrestment of disease.

Another aspect of the present invention is identifying which animals, independent of serological or clinical staging, have, not only systemic dissemination of Map, but also shed the organism into their milk and thus threaten the human food chain. Nesting Map PCR analysis of milk demarcates cows which can remain in production with monitoring rom those who will be slaughtered. Not all infected animal progress to clinical disease (Johne's disease). Like Mycobacterium tuberculosis infection in humans, it is more likely than not, that a given animal's immune system can arrest infection prior to becoming disease. Consistent with this postulate is the inventors ability to convert a clinically diseased dairy cow with Johne's disease back to heath using specific therapy. This approach lessens the adverse economical impact of removing fiscally productive animals from the herd.

Another aspect of this present invention is that it identifies those animals who potentially would benefit most from selective augmentation of their immune system. Low fecal shedders without a negative or low ELISA titer constitute the best candidates for therapeutic rescue and long term survival in the herd.

Another aspect of the present invention is that it identifies pregnant animals who on the basis of heavy shedding are at augmented risk of transmission of Map in utero, but assuredly at parturition through colostrium and milk. Separation of calf from mother at birth may prevent infection transmission at a time of immunological immaturity on the part of the newborn.

Another aspect of the present invention is that it constitutes a comprehensive, logical approach to reducing Map from entering the human food chain while affording some benefits to the herd owners, both financially and legally.

The current USDA guidelines from test-and-cull do not work for Map as it did for brucellosis. To avoid legal liability issues, the commercial Map ELISA tests primarily identify the animals with the most advanced infection/disease The invention constitutes an application of prior invention ingenuity and intellectual property which results in a useful, practical method which has real world significance. Its sequential application can produce useful, concrete and tangible results. The prior art of the inventor has documented the usefulness of individual steps within the proposed method process.

I claim:

1. A method based upon sequential utilization of the diagnostic steps which will reduce the introduction of Map into the human food chain while lessening the adverse economical impact which would result from the total slaughter of all infected animals. The steps comprising
a) screening entire herds or individual subsets of animals using nesting and non-nesting PCR technology to identify animal shedding Map into their feces,
b) using real time PCR technology to identify minimal to low shedders from quantitatively significant shedders,
c) using nesting PCR technology on milk from fecal shedding animals to determine presence of Map DNA

2. The method of claim 1, wherein animals shedding into feces are identified.

3. The method of claim 1, wherein milk containing Map DNA can be identified and separated from milk of fecally shedding Map cow whose milk does not contain Map

4. The method of claim 1, wherein animals fecally shedding Map can, using real time PCR, have the amount of Map being shed quantitated.

5. The method of claim 1, wherein animals with low Map fecal shedding can be identified for enhancement of their immune system

6. The method of claim 1, wherein pregnant animals with high shedding can be handled at parturition so as to minimize the calf's assess to milk or colostrum from that mother.

7. The method of claim 1, wherein using nesting PCR technology on milk identifies animals with systemic dissemination which need to be culled from the herd.

8. The method of claim 1, wherein animals include dairy cows, beef cattle, goats, sheep, buffalo and other ruminants

9. The method of claim 1 is a method by which entire herds of ruminants can be initial screened for infection with Map

10. The method of claim 1 is a method by which system Map infection can be identified
